(19) United States
(12) Patent Application Publication
(10) Pub. No.: US 2008/0249120 A1
(43) Pub. Date: Oct. 9, 2008

(54) Pharmaceutical compositions

(75) Inventors: Vijay Soni, Suffern, NY (US); Vijay Nasare, Navi Mumbai (IN); Ulhas Dhuppad, Maharashtra (IN); Narendra Joshi, Navi Mumbai (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 12/001,807

(22) Filed: Dec. 13, 2007

Related U.S. Application Data
(60) Provisional application No. 60/874,658, filed on Dec. 13, 2006.

(30) Foreign Application Priority Data
Nov. 22, 2006 (IN) 1928/MUM/2006

(51) Int. Cl. A61K 31/505; C07D 239/42

(52) U.S. Cl. 514/275; 544/332

(57) ABSTRACT

A pharmaceutical composition in solid form containing at least a therapeutically effective amount of one or more statins in the free acid form is disclosed.

### PRIORITY

This application claims the benefit under 35 U.S.C. §119 to U.S. Provisional Application No. 60/874,658, filed Dec. 13, 2006, and entitled “PHARMACEUTICAL COMPOSITIONS COMPRISING FREE ACID FORM OF STAINS”, the contents of which are incorporated by reference herein.

### BACKGROUND OF THE INVENTION

1. Technical Field

The present invention relates generally to pharmaceutical compositions containing a stabilized HMG-CoA reductase inhibitor in the free acid form suitable for the treatment of hypercholesterolemia and hyperlipidemia.

2. Description of the Related Art

HMG-CoA reductase inhibitors are a well known class of compounds used to lower cholesterol. Representative examples of such inhibitors include lovastatin (Mevacor), pravastatin (Pravachol), simvastatin (Zocor), fluvastatin (Lescol), atorvastatin (Lipitor), cerivastatin (Baycol) and rosuvastatin (Crestor). Collectively, these compounds are known as “statins”. Statins can lower blood levels of LDL, as well blood fats called triglycerides, but statins also increase blood levels of HDL, known as “good cholesterol.”

Generally, the functional moiety of statins is either a hydroxyl acid in an open non-ring structure with a hydroxyl in the delta position, or a six membered ring closed lactone structure. However, out of these structural arrangements, it is believed that the open hydroxy acid form is the preferred bioactive form and the closed lactone form is apparently the biologically inactive, as the lactone does not seem to inhibit the HMG-CoA reductase enzyme. Several of the statins to be delivered to the gastrointestinal (GI) tract are administered as hydroxyl acid salts, while very few, such as lovastatin and simvastatin, are delivered as closed lactones which are enzymatically hydrolyzed in the body to the apparently active moiety (active metabolite). Pravastatin (U.S. Pat. No. 4,346,227) is administered as the sodium salt. Fluvastatin (U.S. Pat. No. 4,739,073) and cerivastatin (U.S. Pat. No. 5,006,530 and 5,177,080), are also administered as the sodium salt, while atorvastatin and rosuvastatin are administered as calcium salts.

U.S. Pat. No. 5,356,896 discloses a pharmaceutical composition comprising fluvastatin Na and an alkaline stabilizing medium which imparts a pH more than 8 to the pharmaceutical compositions to improve the stability of fluvastatin Na.

U.S. Pat. No. 6,126,971 discloses a pharmaceutical composition comprising atorvastatin in hemicalcium form, and an alkaline earth metal additive salt, preferably calcium carbonate.

U.S. Pat. No. 6,316,460 discloses a pharmaceutical composition comprising rosuvastatin, wherein the stability of rosuvastatin is improved, with an inorganic salt in which a cation is multivalent, preferably tribasic calcium phosphate.

There remains a need to prepare a stabilized active substance of HMG-CoA reductase inhibitor of relatively high purity which will provide lactone contents that will remain stable under normal storage and/or handling conditions and the change of which will be small under storage conditions.

### SUMMARY OF THE INVENTION

In accordance with one embodiment of the present invention, a pharmaceutical composition in a solid dosage form is provided comprising a therapeutically effective amount of one or more statins in the free acid form.

In accordance with a second embodiment of the present invention, a pharmaceutical composition in a solid dosage form is provided comprising a therapeutically effective amount of one or more statins in the free acid form and a pharmaceutically acceptable excipient.

In accordance with a third embodiment of the present invention, a pharmaceutical composition in a solid dosage form is provided comprising a therapeutically effective amount of one or more statins in the free acid form, at least one diluent and optionally an antioxidant and stabilizer.

In accordance with a fourth embodiment of the present invention, a solid composite is provided comprising one or more statins in the form of a free acid, at least one diluent and optionally an antioxidant and stabilizer.

In accordance with a fifth embodiment of the present invention, a pharmaceutical composition is provided comprising a solid composite in admixture with one or more pharmaceutically acceptable excipients, wherein the solid composite comprises a mixture comprising one or more statins in the form of a free acid, at least one diluent and optionally an antioxidant and stabilizer.

In accordance with a sixth embodiment of the present invention, a solid composite is provided comprising a statin in the free acid form and one or more pharmaceutically acceptable excipients, wherein the statin in the free acid form retains at least about 97.5% of its initial purity after two months, and at least about 97% of its initial purity after three months when stored at a temperature of about 2° C. to about 8° C.

In accordance with a seventh embodiment of the present invention, a pharmaceutical composition is provided comprising a statin in the free acid form and one or more pharmaceutically acceptable excipients, wherein the statin in the free acid form retains at least about 97.5% of its initial purity after two months, and at least about 97% of its initial purity after three months when stored at a temperature of about 2° C. to about 8° C.

In accordance with an eighth embodiment of the present invention, a method for the treatment of hypercholesterolemia and hyperlipidemia is provided comprising the step of administering to a patient in need of such treatment a therapeutically effective amount of a pharmaceutical composition comprising a statin in the free acid form and one or more pharmaceutically acceptable excipients, wherein the statin in the free acid form retains at least about 97.5% of its initial purity after two months, and at least about 97% of its initial purity after three months when stored at a temperature of about 2° C. to about 8° C.

### Definitions

The term “solid composite” as used herein is intended to mean a mixture containing at least a statin in the form of a free acid, at least one diluent and optionally an antioxidant and stabilizer in solid form. Generally, solid forms of the “solid composite” can be a powder blend suitable to be incorporated into the pharmaceutical composition of the present invention.

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

The term “subject” or “a patient” or “a host” as used herein refers to mammalian animals, preferably human.

There pharmaceutical compositions of the present invention contain a stabilized active substance of a free acid form of a HMG-CoA reductase inhibitor of high purity and having a lactone content that will remain stable under normal storage and/or handling conditions.

### DETAILED DESCRIPTION OF THE INVENTION

The present invention is directed to pharmaceutical compositions containing at least a therapeutically effective amount of one or more statins in the free acid form. A statin in the form of a free acid represents the basic moiety of statins, not inclusive of metal or amine salt forms in its structure that inhibits HMG-CoA reductase enzyme. The term “statin in the form of free acid” can also be referred to as “statin base”. Representative examples of a statin base for use herein include lovastatin base, pravastatin base, simvastatin base, fluvastatin base, atorvastatin base, cerivastatin base, rosuvastatin base and the like and mixtures thereof. The preferred statin base for use in the pharmaceutical compositions of the present invention is a rosuvastatin base.

The statin bases for use in the present invention can be prepared by a process which intends to prepare a statin base itself and does not include the steps which convert a statin base into a salt form. Alternatively, the statin base herein can be prepared by converting a salt of a statin to its base. In the latter technique, a statin base can be prepared by at least dissolving a statin in a salt form in a suitable solvent followed by neutralization to provide the free statin base. Suitable solvents include water, acetates such as ethyl acetate and the like and mixtures thereof. Neutralization can be carried out by contacting the solution with an amount sufficient to neutralize the solution to a pH of about 3 to about 4.

The neutralization agent can be a suitable acid. Suitable acids include, but are not limited to, hydrochloric acid; sulfonic acid; sulfuric acid; sulfurous acid; phosphoric acid; carbonic acid; saturated or unsaturated C₁-C₄ unsubstituted or halo- or hydroxy-substituted alkanoic mono and di-carboxylic acids such as formic acid, acetic acid, propionic acid, citric acid, tartaric acid, maleic acid, oxalic acid, chloroacetic acid and the like; arylalkanoic acids such as benzoic acid and the like; alkylsulfonic acids such as methanesulfonic acid and the like; aryl sulfonic acids such as p-toluene sulfonic acid and the like; and mixtures thereof. A preferred acid is dilute hydrochloride acid.

The neutralized solution can be contacted with an extracting solvent. Suitable extracting solvents for use herein include acetate-containing solvents and the like and mixtures thereof. A representative example of a useful solvent is ethyl acetate.

The solid residue obtained after the solvent removal may be isolated and dried using conventional methods to provide a statin base.

The pharmaceutical compositions of the present invention can contain one or more pharmaceutically acceptable excipients. Suitable pharmaceutically acceptable excipients comprises at least one non-therapeutic and/or non-toxic pharmaceutical ingredient such as a binder, diluent, disintegrant, surfactant, lubricant and the like and mixtures thereof. Suitable excipients and the amounts to use may be readily determined by the formulation scientist based upon experience and consideration of standard procedures and reference works in the field, e.g., the binders, surfactants, lubricants, and disintegrants described herein.

Suitable binders include, but are not limited to, methyl cellulose, carboxymethylcellulose, hydroxypropylcellulose, hydroxymethyl propylcellulose, polyvinylpyrrolidone, polyvinyl alcohol, starch and the like and mixtures thereof.

Suitable diluents include, but are not limited to, sugar, sugar alcohols, microcrystalline cellulose, hydrous lactose, corn starch, sucrose, silicic anhydride, polysaccharides, N-methyl pyrrolidone (Pharmasolve (ISP)) and the like and mixtures thereof. The term sugar and sugar alcohols comprises mannitol, lactose, fructose, sorbitol, xylitol, maltodextrin, dextrates, dextrins, lactitol and the like and mixtures thereof. The diluent will ordinarily be present in a weight ratio of diluent:statin ranging from about 0.5:1 to about 10:1.

Suitable disintegrants include, but are not limited to, carboxymethylcellulose calcium, croscarmellose sodium, starch and the like and mixtures thereof.

Suitable surfactants include, but are not limited to, Tween 80, polyoxyethylene-polyoxypropylene copolymer and the like and mixtures thereof.

If desired, the pharmaceutical compositions of the present invention may also contain one or more antioxidants in order to prevent any oxidation of the drug compound. Suitable antioxidants for use herein include, but are not limited to, butylated hydroxyanisole, sodium ascorbate, butylated hydroxytoluene, sodium metabisulfate, malic acid, citric acid, ascorbic acid and the like and mixtures thereof. A preferred antioxidant is butylated hydroxyanisole.

If desired, the pharmaceutical compositions of the present invention may also contain one or more stabilizers. Suitable stabilizers for use herein include, but are not limited to, alkaline metal additive salt, metal oxides, metal carbonates and bicarbonates, organic bases and the like and mixtures thereof. Representative examples of useful stabilizers include sodium carbonate, sodium bicarbonate, calcium carbonate, sodium citrate, magnesium oxide, magnesium carbonate, dibasic calcium phosphate, tribasic calcium phosphate, and disodium hydrogen phosphate.

The organic base for use herein includes lysine, N,N′-dibenzylethylenediamine, meglumine, chloroprocain, choline, ethylenediamine and the like and mixtures thereof.

The selection of a solvent for the purpose of present invention depends on the solubility of the particular statin base to be used. In the case of rosuvastatin base, any aliphatic or aromatic hydrocarbon solvent can be used. Representative examples of suitable solvents include hexane, toluene, xylene and the like and mixtures thereof.

The pharmaceutical compositions of the present invention are suitable to deliver one or more statin bases to a human in need of treatment thereof. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, and rectal and transdermal routes. Oral dosage forms include, but are not limited to, tablets, pills, capsules, troches, sachets, suspensions, powders, lozenges and the like.

The pharmaceutical compositions of the present invention can be prepared by incorporating a statin base directly into a pharmaceutical composition, wherein a statin base can be optionally dissolved in any suitable solvent and added to the pharmaceutically acceptable excipients. In one embodiment, the statin base is first converted to a solid composite as discussed hereinbelow before incorporating into a pharmaceutical composition.

In another embodiment, the pharmaceutical compositions of the present invention can be prepared by (a) providing a mixture by adding a statin base to a diluent such as sugar, sugar alcohol or microcrystalline celluloses, silicon dioxide and combinations thereof, optionally mixed with a binder, disintegrant, antioxidant and/or stabilizer; and (b) subjecting the mixture to dry granulation or direct compression. The powder/granules thus obtained can be lubricated with a suitable lubricant and filled into capsules or compressed into tablets. If the obtained mixture is a wet mass, it can be subjected to drying, milling and lubrication before filling into a capsule or compressing into tablets. Representative examples of a suitable lubricant include, by way of example and without limitation, calcium stearate, magnesium stearate, mineral oil, stearic acid, zinc stearate, and the like and combinations thereof.

In another embodiment, the pharmaceutical compositions of the present invention can be prepared by (a) dissolving or dispersing a statin base in a suitable solvent, e.g., ethyl acetate or diethyl ether for a rosuvastatin base, optionally containing an antioxidant and/or stabilizer; (b) adding a diluent, optionally mixed with a binder and/or disintegrant, to the solution or dispersion thus obtained; (c) drying the wet mass; and (d) blending the dried mass to prepare capsules or tablets. In one embodiment, the wet mass can be extruded to obtain ribbon like extrudes, which can then be subjected to a spheroniser to convert the extrudes to spherical beads or pellets or spheroids.

In another embodiment, the pharmaceutical compositions of the present invention can be prepared by (a) dissolving or dispersing a statin base in a solution containing a binder and optionally an antioxidant and/or stabilizer; (b) adding to the solution or dispersion a diluent, such as sugar, sugar alcohol or microcrystalline celluloses, silicon dioxide or combination thereof, and optionally mixed with a binder and/or disintegrant; (c) drying the wet mixture and (d) lubricating the dried mass.

If desired, a stabilizer can be added at any stage, i.e., a stabilizer can be added in solution or it can be mixed with a diluent or with the dried granules.

In another embodiment, a solid composite of the present invention can be prepared by spray drying a solution or suspension comprising a statin base in a suitable solvent, e.g., ethyl acetate or diethyl ether for a rosuvastatin base, and optionally a diluent, an antioxidant and/or stabilizer. In one embodiment, a solid composite of the present invention can be prepared by spray drying a solution or suspension comprising a statin base, and a diluent and optionally an antioxidant and/or stabilizer. The solid composite obtained in the form of spray dried particles can be incorporated into a pharmaceutical composition of the present invention.

In another embodiment, a solid composite of the present invention can be prepared by spraying a solution or suspension comprising a statin base in a suitable solvent, e.g., ethyl acetate or diethyl ether for a rosuvastatin base, and optionally an antioxidant and/or stabilizer, in a fluid bed coater, over a diluent such as sugars, sugar alcohols, saccharides, silicon dioxides, celluloses and the like and mixtures thereof. If desired, the diluent can be mixed with a disintegrant, antioxidant and stabilizer prior to being sprayed with the solution. The solid composite thus obtained can be incorporated into a pharmaceutical composition of the present invention.

In another embodiment, a solid composite of the present invention can be prepared by (a) dissolving a statin base in a suitable solvent, e.g., ethyl acetate or diethyl ether for a rosuvastatin base; (b) adding a diluent such as a sugar, sugar alcohol, saccharide, silicon dioxide, cellulose, N-methyl pyrrolidone and the like and mixtures thereof to the solution; (c) distilling the solvent from the mixture; and (d) degassing the material to obtain a solid composite.

The present invention is further illustrated by the following examples which are provided merely to be exemplary of the invention and do not limit the scope of the invention.

### EXAMPLE 1

Preparation of Rosuvastatin base from Rosuvastatin Calcium

Rosuvastatin calcium (10 g) was added to water (100 ml), and ethyl acetate (50 ml). The pH of the solution was adjusted to 3.5 to 3.7 using a dilute hydrochloric acid at 20-25° C. The rosuvastatin base was extracted from the solution using 3×50 ml of ethyl acetate. The ethyl acetate layer was combined with brine solution and was dried over sodium sulfate. The ethyl acetate layer was subjected to reduced pressure at 25 to 30° C. to provide rosuvastatin base which had an oily consistency.

### EXAMPLE 2

Preparation of a Tablet

Rosuvastatin base (200 g) was dissolved in a sufficient quantity of isopropyl alcohol. The solution of rosuvastatin base was added to a mixture containing lactose (560 g), microcrystalline cellulose (695 g) and crospovidone (75 g). The wet mass thus obtained was dried and the dried granules were lubricated with magnesium stearate (7.5 g). The lubricated granules were compressed into 5000 tablets.

### EXAMPLE 3

Preparation of a Tablet

Rosuvastatin base (200 g) was dissolved in a sufficient quantity of isopropyl alcohol. The solution of rosuvastatin base was added to a mixture containing lactose (560 g), microcrystalline cellulose (695 g), meglumine (12.5 g), butylated hydroxy toluene (0.25 g) and crospovidone (75 g). The wet mass thus obtained was dried and the dried granules were lubricated with magnesium stearate (7.5 g). The lubricated granules were compressed into 5000 tablets.

### EXAMPLE 4

Preparation of a Tablet

Rosuvastatin base (200 g) was dissolved in a solution of PVP (20 g) in a sufficient quantity of isopropyl alcohol. The solution of rosuvastatin base was added to a mixture containing lactose (560 g) and microcrystalline cellulose (695 g), meglumine (12.5 g), butylated hydroxy toluene (0.25 g) and crospovidone (75 g). The wet mass thus obtained was dried and the dried granules were lubricated with magnesium stearate (7.5 g). The lubricated granules were compressed into 5000 tablets.

### EXAMPLE 5

Preparation of a Tablet

Rosuvastatin base (200 g) was dissolved in a solution of PVP (15 g) in sufficient quantity of isopropyl alcohol. The solution of rosuvastatin base was sprayed over a mixture containing mannitol (600 g) crospovidone (30 g) and meglumine (50 g) in a fluidized bed coater. The wet mass thus obtained was dried and the dried granules were mixed with crospovidone (40 g) and lubricated with magnesium stearate (14 g). The lubricated granules were compressed into 5000 tablets.

### EXAMPLE 6

Preparation of a Tablet

Rosuvastatin base (2.5 g) was dissolved in diethyl ether (50 ml). Mannitol (PEARLITOL® 200 SD) (5 g) was added to the solution at 25 to 30° C. and the mixture was stirred for 1 hour. The solvent was evaporated under reduced pressure at 25 to 30° C. and the gases were removed from the product. The product was withdrawn from the flask, to obtain 7.5 g of a solid composite. The solid composite thus obtained was mixed with microcrystalline cellulose (400 g), meglumine (50 g) and crospovidone (55 g). The blend was further mixed with magnesium stearate (15 g) and compressed into tablets.

### EXAMPLE 7

Preparation of a Tablet

Rosuvastatin base (2.5 g) was dissolved in diethyl ether (50 ml). Colloidal silicon dioxide (Aerosil-200) (2.5 g) was added to the solution at 25 to 30° C. and the mixture was stirred for 1 hour. The solvent was evaporated under reduced pressure at 25 to 30° C. The gases were removed from the product and the product was withdrawn from the flask, to obtain 7.5 g of a solid composite. The solid composite thus obtained was mixed with microcrystalline cellulose (400 g), meglumine (50 g) and crospovidone (55 g). The blend was further mixed with magnesium stearate (15 g) and compressed into tablets.

### EXAMPLE 8

Preparation of a Tablet

Rosuvastatin base (2.5 g) was dissolved in 50 ml of diethyl ether. Microcrystalline cellulose-NF (Avicel pH 102) (5 g) was added to the solution at 25 to 30° C. and the mixture was stirred for 1 hour. The solvent was evaporated under reduced pressure at 25 to 30° C. The gases were removed from the product and the product was withdrawn from the flask, to obtain 7.5 g of a solid composite. The solid composite thus obtained was mixed with microcrystalline cellulose (400 g), meglumine (50 g) and crospovidone (55 g). The blend was further mixed with magnesium stearate (15 g) and compressed into tablets.

### EXAMPLE 9

Preparation of a Tablet

Rosuvastatin base (2.5 g) was dissolved in 50 ml of diethyl ether. Mannitol (PEARLITOL® 200 SD) (5 g) and meglumine (1 g) was added to the solution at 25 to 30° C. and the mixture was stirred for 1 hour. The solvent was evaporated under reduced pressure at 25 to 30° C. The gases were removed from the product and the product was withdrawn from the flask, to obtain 7.5 g of a solid composite. The solid composite thus obtained was mixed with microcrystalline cellulose (400 g), and crospovidone (55 g). The blend was further mixed with magnesium stearate (15 g) and compressed into tablets.

### EXAMPLE 10

Preparation of a Capsule

Rosuvastatin base (200 g) was dissolved in a sufficient quantity of isopropyl alcohol. The solution of rosuvastatin base in isopropyl alcohol was added to the mixture comprising lactose, lysine and microcrystalline cellulose. The wet mass thus obtained was subjected to an extruder and then a spheronizer to obtain spherical pellets. The pellets were filed into capsules.

### EXAMPLE 11

Rosuvastatin tert-butyl amine salt (50.0 g) was added to water (300.0 ml) and diethyl ether (300.0 ml). The pH was adjusted to 3.0 to 3.5 with 10% v/v dil. hydrochloric acid at 0 to 5° C. The free acid was extracted with 2×50.0 ml of diethyl ether. The diethyl ether layers were combined and washed with a brine solution and dried over sodium sulfate. The solution and sodium sulphate were filtered and washed with 50.0 ml diethyl ether. The total volume of the diethyl ether layer was 445.0 ml. The diethyl ether was distilled out completely under 0 to 10 torr vacuum at 20 to 25° C. After degassing completely, rosuvastatin free acid (40.0 g) was isolated. Next, the rosuvastatin free acid was dissolved in Pharmasolve (40.0 ml) at 0 to 5° C. The solvent was further evaporated and a solid composite was produced.

The solid composite was stored at 2 to 8° C. for three months. The % purity of the rosuvastatin free acid/pharmasolve solid composite was determined high performance liquid chromatography (HPLC) analysis. The results are set forth below in Table 1.

TABLE 1

| Time | % Purity | Lactone content |
|---|---|---|
| Initial | 99.60% | 0.07% |
| After 2 months | 97.70% | 2.10% |
| After 3 months | 97.30% | 2.50% |

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A pharmaceutical composition in solid form comprising a therapeutically effective amount of one or more statins in the free acid form.

2. The pharmaceutical composition of claim 1, wherein the statin in the free acid form is rosuvastatin free acid.

3. The pharmaceutical composition of claim 1, further comprising one or more pharmaceutically acceptable excipients.

4. The pharmaceutical composition of claim 2, comprising one or more pharmaceutically acceptable excipients.

5. The pharmaceutical composition of claim 1, comprising a solid composite comprising a therapeutically effective amount of the one or more statins in the free acid form and at least one pharmaceutically acceptable excipient.

6. The pharmaceutical composition of claim 5, wherein the pharmaceutically acceptable excipient is N-methylpyrrolidone.

7. A pharmaceutical composition in solid form comprising a statin in the free acid form and one or more pharmaceutically acceptable excipients, wherein the statin in the free acid form retains at least about 97.5% of its initial purity after two months, and at least about 97% of its initial purity after three months when stored at a temperature of about 2° C. to about 8° C.

8. The pharmaceutical composition of claim 7, wherein the pharmaceutically acceptable excipient is a diluent.

9. The pharmaceutical composition of claim 7, wherein the statin in the free acid form is rosuvastatin free acid.

10. The pharmaceutical composition of claim 7, wherein the pharmaceutically acceptable excipient is selected from the group consisting of sugar alcohol, disaccharide, N-methylpyrrolidone, colloidal silicon dioxide, cellulose derivative and mixtures thereof.

11. The pharmaceutical composition of claim 7, wherein the pharmaceutically acceptable excipient is N-methylpyrrolidone.

12. The pharmaceutical composition of claim 7, having a content of the lactone form of the rosuvastatin free acid of no more than about 3%.

13. The pharmaceutical composition of claim 7, having a content of the lactone form of the rosuvastatin free acid of no more than about 2.5%.

14. A solid composite comprising rosuvastatin in the free acid form and one or more pharmaceutically acceptable excipients, wherein the rosuvastatin in the free acid form retains at least about 97.5% of its initial purity after two months, and at least about 97% of its initial purity after three months when stored at a temperature of about 2° C. to about 8° C.

15. The solid composite of claim 14, wherein the pharmaceutically acceptable excipient is a diluent.

16. The solid composite of claim 14, wherein the pharmaceutically acceptable excipient is N-methylpyrrolidone.

17. The solid composite of claim 14, having a content of the lactone form of the rosuvastatin free acid of no more than about 3%.

18. The solid composite of claim 14, having a content of the lactone form of the rosuvastatin free acid of no more than about 2.5%.

19. A process for preparing a free acid form of rosuvastatin, the process comprising (a) dissolving rosuvastatin salt into one or more solvents; (b) adjusting the pH of the solution to about 3.0 to about 3.5; and (c) isolating the free acid of rosuvastatin.

20. The process of claim 19, wherein the solvent is diethyl ether or ethyl acetate.

21. The process of claim 19, wherein the step of isolating comprises extracting the free acid form of rosuvastatin from the solution.
